(19) United States
(12) Patent Application Publication
(10) Pub. No.: US 2013/0066255 A1
(43) Pub. Date: Mar. 14, 2013

(54) IONTOPHORETIC PATCH WITH SIDE TAB

(71) Applicants: Michael K. Jordan, North Smithfield, RI (US); Lynn T. Couchon, North Kingston, RI (US); Cuixia Liu, Warwick, RI (US); Miaoyong Cao, Warwick, RI (US)

(72) Inventors: Michael K. Jordan, North Smithfield, RI (US); Lynn T. Couchon, North Kingston, RI (US); Cuixia Liu, Warwick, RI (US); Miaoyong Cao, Warwick, RI (US)

(73) Assignee: Isis Biopolymer, Inc., Providence, RI (US)

(21) Appl. No.: 13/543,366

(22) Filed: Jul. 6, 2012

Related U.S. Application Data
(60) Provisional application No. 61/504,969, filed on Jul. 6, 2011.

(51) Int. Cl. A61N 1/30

(52) U.S. Cl. 604/20

(57) ABSTRACT

The present disclosure relates to an iontophoretic device having a delivery portion and a control portion that are located on a common base layer but separated from one another so as to provide increased flexibility and interface with a wearer.

### RELATED APPLICATION

This application claims priority to U.S. Provisional Application Ser. No. 61/504,969 filed 6 Jul. 2011, the entirety of which is incorporated herein by reference.

### FIELD OF THE INVENTION

The invention relates to the field of devices for transdermal delivery of drugs to a patient, and more particularly to devices wherein certain control components are isolated from certain delivery components while sharing a common base with those components.

### BACKGROUND OF THE INVENTION

Transdermal delivery devices are known in the art. For example, iontophoretic devices can be used for transdermal delivery of drugs. These devices are placed on a patient's skin and use charged electrodes to drive charged drug ions from a drug reservoir into the patient's skin tissue.

Current self-contained transdermal patch technology suffers from certain limitations such as, for example, the presence of moisture and other substances near electronic control components during drug delivery. Typical solutions to these limitations include additional complexity in patch design to shield control components from such undesirable substances. Current self-contained patch systems also suffer from limitations relating to thickness, flexibility, and wearability due to the presence of control components that resist flexibility or are damaged by flexing forces.

The systems and devices provided herein provide solutions to these and other problems in the art.

### SUMMARY OF THE INVENTION

The invention provides for self-contained transdermal drug delivery devices. In some implementations, a patch is provided to deliver drugs into the tissue of a wearer. The patch may include a delivery portion and a control portion. In some implementations, the delivery portion may include or house a drug reservoir, a driving electrode, a counter electrode, adhesive portions, and/or other elements. In some implementations, the control portion may include or house a controller, a power source, and/or other elements. In some implementations, the control portion may be attached to the delivery portion by a bridge portion. In this implementation, the control portion may exist as a “side-tab” of the device that is apart from, but attached to the delivery portion. As described below, in some implementations, this side-tab configuration may include the delivery portion having adhesive so that it can adhere to the skin of a wearer and the control portion not including the adhesive. In this manner, the control portion may protrude from the skin of the wearer while being attached to the delivery portion. Thus, the control portion may enable removal of the patch from the skin of a wearer, as control portion may serve as a “pull tab” that a wearer's hand (or other person's hand) may grasp to remove the patch.

Furthermore, locating certain elements of the patch on the control portion, separate from the delivery portion, enables those elements that hinder flexibility to be separated from the portion of a patch that is engaged with the tissue of a patient, therefore providing improved contact of delivery-related elements with such tissue. The separation also assists in keeping moisture and other disruptive substances away from such control-related elements and obviates the need for numerous through-holes in the structure of the patch.

In some implementations, the control portion, bridge portion, and delivery portion may include a base layer wherein all electrodes, control elements, and circuitry are located only on a bottom (or patient-tissue-facing) side of the base layer, therefore avoiding the need for any through-holes in the patch. As certain control elements are located on the control portion, which does not adhere to patient tissue, the problem of disruption of patch operation from moisture and other substances is avoided.

Other objects, features, and advantages of the present application will become apparent from the following detailed description, the accompanying drawings, and the appended claims.

### BRIEF DESCRIPTION OF THE DRAWINGS

FIG. 1 is an example of an iontophoretic patch according to various embodiments of the invention.

FIG. 2A is an exploded view of an iontophoretic patch according to various embodiments of the invention.

FIG. 2B is a bottom cross-sectional view of an iontophoretic patch according to various embodiments of the invention.

FIG. 2C is a close-up bottom cross-sectional view of a control portion and bridge portion of an iontophoretic patch according to various embodiments of the invention.

FIG. 3 is an example of an iontophoretic patch according to various embodiments of the invention.

FIG. 4 is an example of an iontophoretic patch according to various embodiments of the invention.

FIG. 5 is an example of an iontophoretic patch according to various embodiments of the invention.

FIG. 6 is an example of an iontophoretic patch according to various embodiments of the invention.

### DETAILED DESCRIPTION OF THE ILLUSTRATED EMBODIMENT(S) OF THE INVENTION

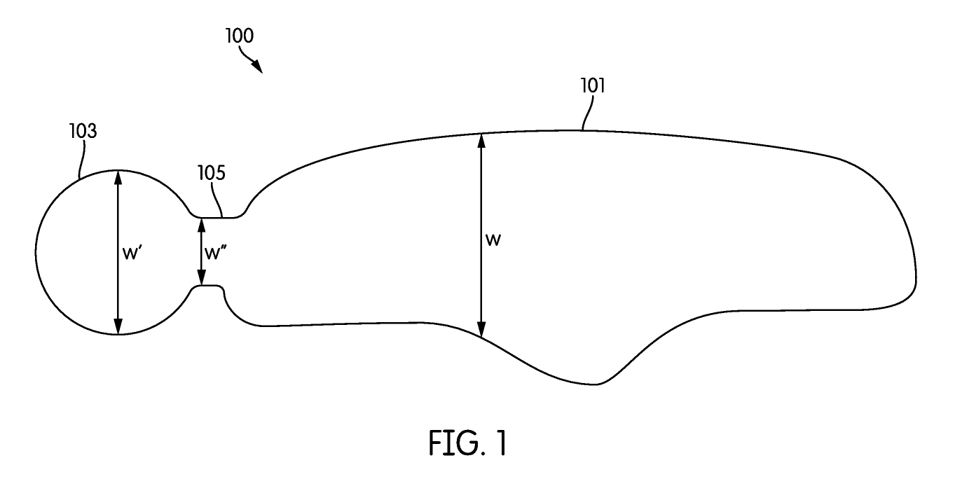

FIG. 1 illustrates an iontophoretic patch 100, which is an example of an iontophoretic delivery device according to various implementations of the invention. Patch 100 is configured to deliver drugs into the tissue of a wearer. The basic principles of iontophoretic devices are well-known, as described, for example, in U.S. Patent Publication No. 2009/0048556 and U.S. Patent Publication No. 2009/0299267, both of which are hereby incorporated by reference herein in their entirety.

Patch 100 may include a delivery portion 101 and a control portion 103. In some implementations, delivery portion 101 may include or house a drug reservoir, a driving electrode, a counter electrode, adhesive portions, and/or other elements. In some implementations, control portion 103 may include or house a microprocessor-based controller, a power source, and/or other elements. In some implementations, control portion 103 may be attached to delivery portion 101 by a bridge portion 105 that has a width (w″, which may be, for example, 0.500 inches) narrower than the width of both the delivery portion (w, which may be, for example, 1.345 inches or 2.096 inches at the center of the delivery portion) and the control portion (w′, which may be, for example, 1.350 inches). In this implementation, control portion 103 may exist as a “side-tab” of the device that is apart from, but attached to delivery portion 101. The side tab can be located on either side, top, or bottom of delivery portion 101. That is, the term “side tab” refers to a tab extending from any side, including the top or bottom, and not just a lateral side. As described herein, in some implementations, this side-tab configuration may include delivery portion 101 having adhesive so that it can adhere to the skin of a wearer and control portion 103 not including the adhesive. In this manner, control portion 103 may protrude from the skin of the wearer while being attached to delivery portion 101. Thus, the control portion may enable removal of patch 100 from the skin of a wearer, as control portion may serve as a “pull tab” that a wearer's hand (or other person's hand) may grasp to remove patch 100.

Locating certain elements of patch 100 on control portion 103 that is separate from delivery portion 101 enables those elements that hinder flexibility of an iontophoretic patch to be separated from the portion of a patch that is engaged with the tissue of a patient, therefore providing improved contact of crucial delivery-related elements with such tissue. In fact, as described herein, control portion 103 may be completely free of adhesive, such that those flexibility-hindering elements are not on a portion of the device that contacts or is adhered to a wearer's tissue. Furthermore, the separation of certain control-related elements from the tissue of the wearer assists in keeping moisture and other disruptive substances away from such control-related elements and obviates the need for numerous through-holes in the structure of the patch.

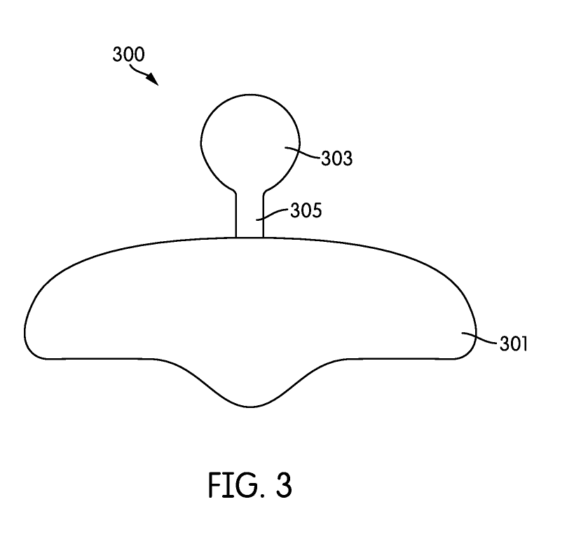

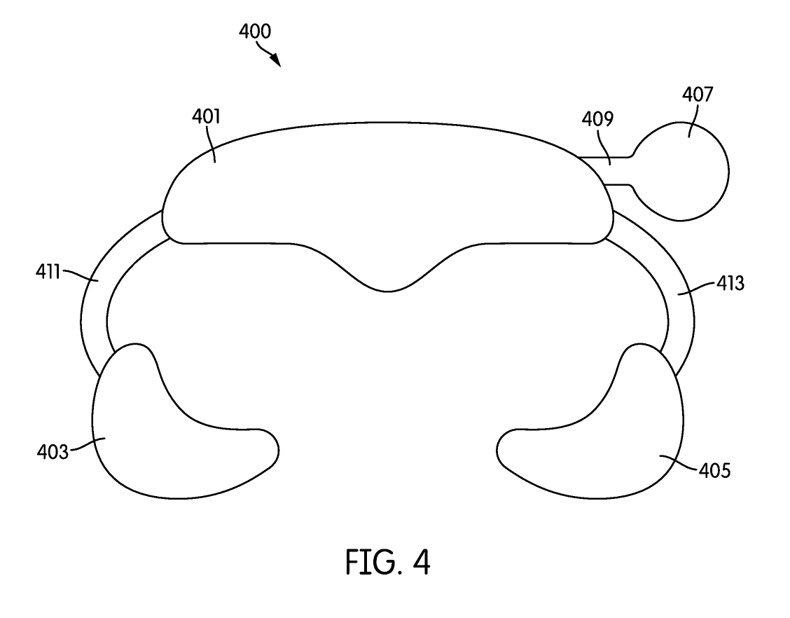

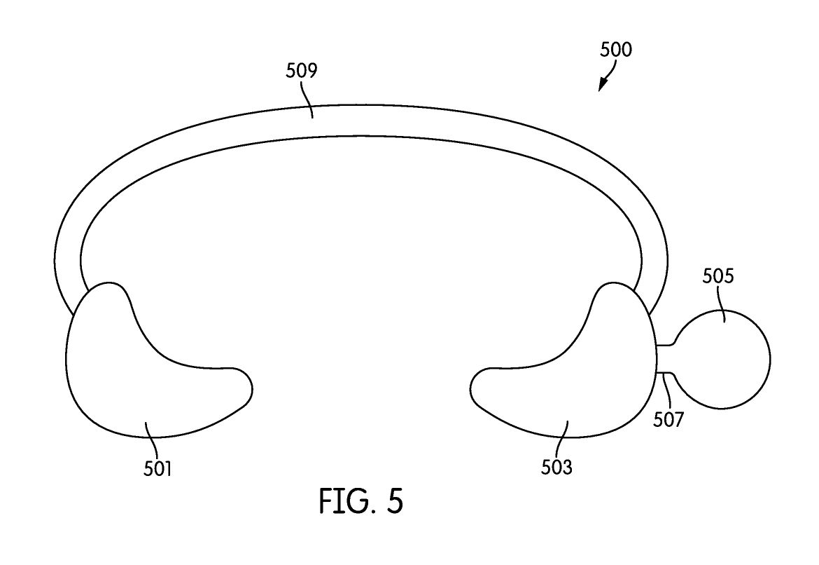

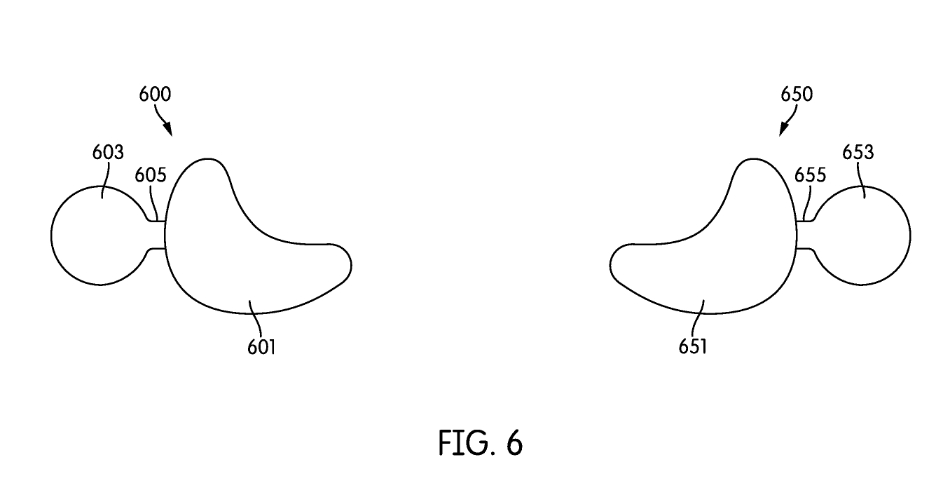

While delivery portion 101 of patch 100 is illustrated as configured as a brow patch (e.g., to be worn on the forehead of a user), other configurations may be used. FIGS. 3-6 illustrate examples of other configurations for a patch according to various implementations of the invention. FIG. 3 illustrates a patch 300, which includes a delivery portion 301 shaped as a brow patch, a control portion 303 located on the top of delivery portion 301, and a bridge portion 305. FIG. 4 illustrates a patch 400, which includes delivery portions 401 (shaped as a brow patch), 403 (shaped as a crow's foot/under eye patch), and 405 (shaped as a crow's foot/under eye patch). Patch 400 also includes control portion 407 located on a side of delivery portion 401 and a bridge portion 409 connecting control portion 407 to delivery portion 401. Patch also includes connector portions 411 and 413 that connect delivery portions 403 and 405 to delivery portion 401, respectively. Connector portions 411 and 413 may include electrical connectors so that delivery portions 411 and 413 are controlled by control portion 407 and therefore behave similarly to delivery portion 401. FIG. 5 illustrates a patch 500, which includes delivery portions 501 and 503, shaped as crow's foot/under eye patches, a control portion 505 (located on a side of delivery portion 503), a bridge portion 507 (connecting control portion 505 to delivery portion 503) and a connector portion 509 (operatively connecting delivery portion 501 to delivery portion 503 and the remainder of patch 500). FIG. 6 illustrates eye patches 600 and 650, both of which are configured as crow's foot/under-eye patches. Patch 600 includes a delivery portion 601, a control portion 603, and a bridge portion 605. Patch 650 includes a delivery portion 651, a control portion 653, and a bridge portion 655. Other configurations may also be used.

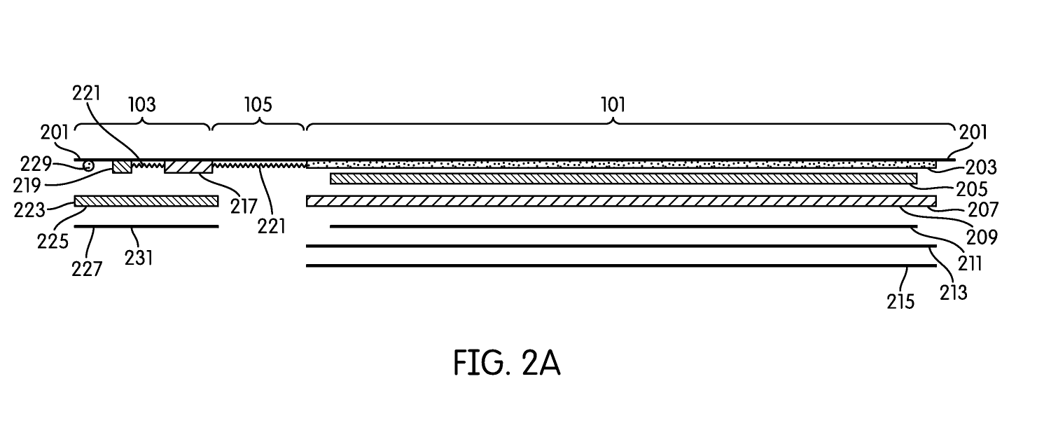

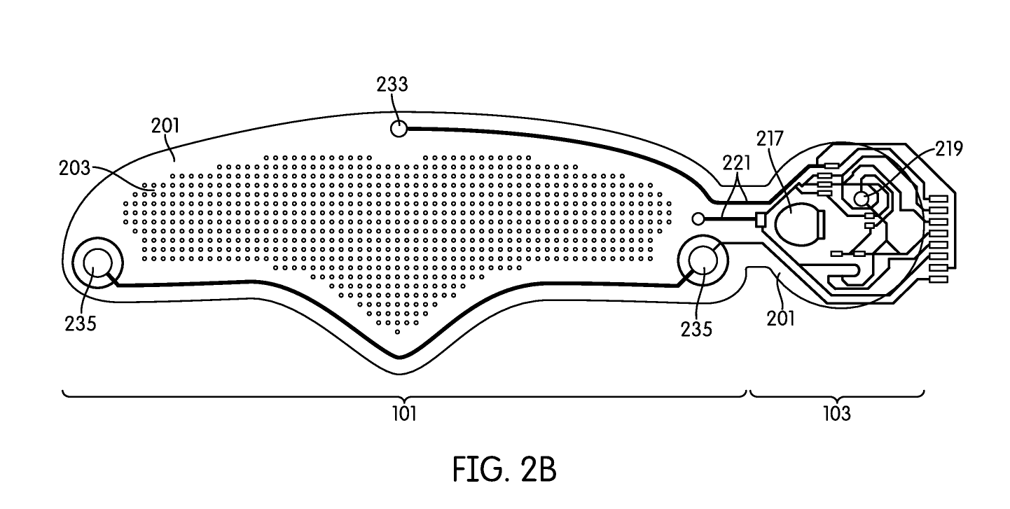

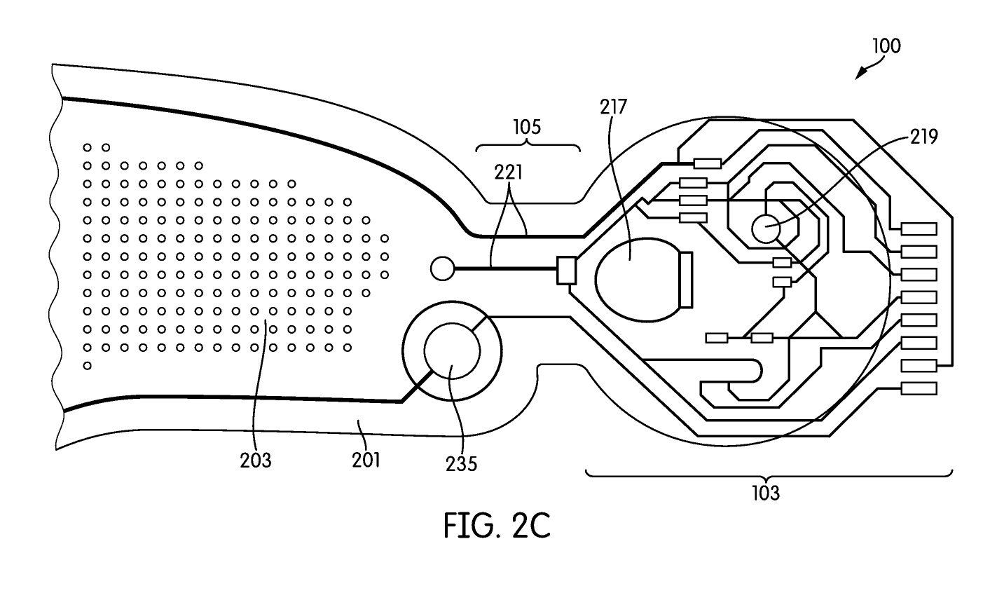

FIGS. 2A, 2B, and 2C illustrate an example of constituent components of patch 100, according to various implementations. Patch 100 may include a base layer 201. Base layer 201 may be a flexible planar structure or layer, such as a thin plastic substrate, that conforms to the body of the patient. In some implementations, base layer 201 may be formed of a dielectric (i.e., electrically insulative) substrate, such as a flexible non-conductive polymer substrate. As illustrated in FIG. 2A, base layer 201 may be present in delivery portion 101, control portion 103, and bridge portion 105. That is, base layer 201 is a continuous and integral piece that connects delivery portion 101, control portion 103, and bridge portion 105 as one unit.

In some implementations, a driving electrode 203 may be attached to or printed only on the bottom surface of base layer 201 on delivery portion 101. Driving electrode 203 may also be referred to in the art as a donor electrode. Driving electrode 203 may be constructed from silver ink, silver chloride ink, and/or a combination of the two. In some implementations, a reservoir 205 may also be located on delivery portion 101. In some implementations, reservoir 205 contains a supply of charged drug ions, which may be elemental ions (i.e., the ionic form of an element), molecular ions (i.e., the ionic form of a molecule), or complexed ions (i.e., ions of a weakly bonded group of elements/molecules/ions referred to as a complex). In some implementations, reservoir 205 comprises a gel, such as a hydrogel. The drug may be solvated in a solution in charged ionic form along with the polymer for the gel, and upon curing, the polymer cross-links and the charged drug ions are stored in the gel. For example, a salt of the drug may be dissolved in the solution, thus providing drug ions with mobility within the ionically conductive solution/gel. The methods by which such drug reservoirs are formed are known and need not be detailed herein. For example, reservoir 205 may simply be the gel, or it may have a more complex structure, such as a partitioned reservoir with an internal membrane for separating and managing ion mobility. Reservoir 205 may have any construction or configuration, and the illustrated embodiment is not limiting.

The term drug may include any bioactive agent, such as pharmaceuticals, vitamins, treatments, elements, etc., and is not limited to just those drugs subject to regulatory approval. As such, the term drug should be interpreted as meaning any agent having a biological effect on the wearer that is transdermally delivered by the device.

In some implementations, a spacer layer 207 may be located on delivery portion 101 beneath driving electrode 203. Spacer layer 207 may exist along the edge of base layer 201/driving electrode so as to define a hole 209 in which reservoir 205 is located. In some implementations, spacer layer 207 may be constructed of a foam or other cushioned material of sufficient thickness as to allow reservoir 205 to be located within hole 209.

In some implementations, a barrier layer 211 is positioned below spacer layer 207/reservoir 205 so as to be positioned between reservoir 205 and the tissue of the wearer. In some implementations, barrier layer 211 has the same configuration as or is larger than reservoir 205 in terms of area. That is, barrier layer 211 may cover the entirety of reservoir 205, thus maintaining its position between reservoir 205 and the wearer's skin. In some implementations, barrier layer 211 is configured to essentially prevent passive transport of the charged drug molecules therethrough.

In some implementations, barrier layer 211 is a mesh. The mesh may be coated with an electroconductive material, such as, for example, Ag, AgCl, or carbon. The coating may vary based on the specific drug molecule, delivery rate, and other requirements. The mesh may have any pore size, such as, for example, between 7 and 100 microns. The pore size may also vary depending on the specific drug molecule, delivery rate, and other requirements.

In some implementations, an adhesive layer 213 may be coated about the peripheral edge of base layer 201 on delivery portion 101 such that adhesive layer 213 is positioned beneath spacer layer 207. In some implementations, adhesive layer 213 may be horizontally integrated with barrier layer 211. In some implementations, the adhesive is a high tack adhesive that firmly bonds delivery portion 101 against a patient's skin. By extending the adhesive to the peripheral edge of delivery portion 101, the adhesive serves to discourage lifting or peeling of the edges of delivery portion 101, thus maintaining it securely fastened to the skin. Other suitable attachment methods may be used to secure the device to a patient, such as tape, straps, etc. In some implementations, as discussed herein, no adhesive is used on bridge portion 105 or control portion 103. Accordingly, the side tab of control portion 103 does not adhere to the patient's skin and therefore may protrude from or hang off of the patient while remaining attached to delivery portion 101 by bridge portion 105. Because control portion 103 does not adhere to the skin of the patient, it need not be as flexible as delivery portion 101. As such, control portion 103 is suited to include or house those elements of an iontophoretic patch that are not conducive to flexibility and which may be damaged by substances found on the tissue of the patient, such as, for example, a controller, a power source, and/or other elements.

In some implementations, delivery portion 101 may include a release liner or embossed cover 215 that covers the adhesive layer 213. In some implementations, release liner 215 covers the entire bottom surface of delivery portion 101 such that release liner 215 covers both the adhesive layer 213 and the bottom of barrier layer 211. Release liner 215 may be paper, plastic or another material, and the upper side of release liner 215 may have a release material, such as silicone or wax, so it can be peeled off to expose adhesive layer 213 and the bottom of barrier layer 211.

In some implementations, control portion 103 may include a power source such as, for example, a battery 217, located only on the bottom surface of base layer 201. In some implementations, battery 217 may be of a printed type (see e.g., U.S. Patent Publication No. 2009/0048556), although any type of battery/power source may be used.

In some implementations, control portion 103 may include a microprocessor 219 or other controller that is coupled to circuitry and to battery 217 for controlling the delivery of electrical power. Collectively, the circuitry, microprocessor 219, and battery 217 may be considered a “control circuit” that controls the application of potentials to the electrodes used in patch 100 (described below). Although a microprocessor is preferred for precise control of the potentials applied to the electrodes described herein, it may be omitted and the control may be provided by basic circuit elements as well. For example, in some implementations, microprocessor 219 may be omitted, and switches may be used in the control circuit (i.e., as a controller) for controlling current flow/direction and the application of the various potentials to the electrodes. Thus, the term control circuit is a structural term that encompasses any circuit coupled to the electrodes for applying potentials thereto, including circuits with or without a microprocessor, integrated circuits, and/or switch-operated circuits.

In some implementations, driving electrode 203, battery 217, and microprocessor 219 may be operatively connected to one another via circuitry 221, thus controlling the application of electrical potential from battery 217 to driving electrode 203. In some implementations, circuitry 221 is printed or coated only on the bottom of base layer 201. An example of that coating technique can be found in the above-referenced U.S. Patent Publication 2009/0048556. One example of a flexible electroconductive material that can be used for printing circuitry 221, electrode 203 or other portions of patch 100 is silver conductive ink (e.g., having a resistivity of 8 to 10 milliohms per square). The resistivity of silver conductive ink within the range of 8 to 10 milliohms per square may be desirable in order to have sufficient current to drive drugs into the stratum corneum of a wearer. The ink may be silver (Ag), silver chloride (AgCl), or other material, for example, and may be printed (e.g., by screen printing or gravure rolling) onto base layer 201.

As driving electrode 203, battery 217, microprocessor 219, and circuitry 221 are all located entirely on the bottom side of base layer 201 with no electrical or other components located on an upper surface of base layer 201, there is no need for a through-hole or other passageway to connect the electrical components of patch 100. This simplifies manufacturing and use, as well as providing a more reliable and flexible design. As the battery and microprocessor are located on control portion 103 fashioned as a “side tab,” which is not adhered to the skin or tissue of the wearer, these components are protected from fouling or other interference from moisture and unwanted substances, even though they are located only on the underside of base layer 201.

As described herein, driving electrode 203 is positioned above reservoir 205 opposite barrier layer 211. In some implementations, driving electrode 203 has the same size (or larger) and configuration as reservoir 205 and the opening 209 in spacer layer 207 in terms of area, thus enabling the potential applied to driving electrode 203 to be exposed to the entirety of reservoir 205. During operation in a driving mode, the control circuit applies a potential to driving electrode 203 of the same polarity as the charge of the charged drug ions so as to drive the charged drug ions towards and into the tissue of the wearer. That is, if the drug is in the form of a positively charged ion, driving electrode 203 will have a positive charge applied to it. Because charges of the same polarity repel, the positively charged drug ions will be repelled away from driving electrode 203 and driven towards the tissue of the wearer's skin for permeation into the skin. Conversely, if the drug ion is in the form of a negatively charged ion, driving electrode 203 will have a negative charge applied to it, thus similarly repelling and driving the drug ions.

In some implementations, barrier layer 211 is configured to permit the charged drug ions to be actively transported therethrough in the driving mode via the potential applied to driving electrode 203. That is, barrier layer 211 may be constructed such that it normally prevents passive transport of the charged drug molecules, but allows the active driven transport by the electromotive force of driving electrode 203 to occur.

In some implementations, barrier layer 211 may be formed of an electroconductive material and is also coupled to the control circuit by circuitry 221 that is located entirely on the bottom side of base layer 201. As such, in some implementations, the control circuit is configured to apply a potential of opposite polarity to barrier layer 211 so that the barrier layer functions as a counterelectrode. That is, the potential of driving electrode 203 is applied from one terminal of the power source, whereas the potential of barrier layer 211 functioning as a counterelectrode is applied from the opposite terminal of the power source. The term counterelectrode specifically refers to and means the electrode that is counter or opposite in charge to driving electrode 203 and is provided for purposes of completing the iontophoretic circuit between the connections to the opposing terminals of the power source. Microprocessor 219 of the control circuit may be configured to control the application of potentials to both electrodes, and various circuit elements may be used to determine the potential and current density applied to each electrode to ensure proper delivery of the drug molecules.

In some implementations, a gel of reservoir 205 is electroconductive, thus completing the circuit comprising driving electrode 203 and barrier layer/counterelectrode 211. The gel may have sufficiently high resistance to maintain a sufficiently high potential difference between the electrodes. In some implementations, rather than rely on the drug reservoir for electroconductively coupling the electrodes, a resistor or other element with a level of resistance may be used to enable the current flow between the electrodes while maintaining an adequate potential difference between the electrodes.

As an example, lidocaine contained in a water-based gel can be delivered using a current density of 0.2 mA/cm² (assuming driving electrode 203 and counterelectrode/barrier layer 211 have the same area). Other examples of drugs that can be used with the invention include hyaluronic acid and acetyl hexapeptide-8, which can be delivered using a current density of 0.5 mA/cm². Other drugs or substances can also be used.

Because barrier layer 211, when used as a counterelectrode, will have the opposite polarity as the charged drug molecules in the driving mode, this may enhance the transport of the drug ions. This is because the charged drug molecules will be both repelled away from driving electrode 203, and attracted towards barrier layer 211. This may beneficially increase the rate of drug transport achieved per unit power, since both electrodes are contributing to drug transport in the same direction towards the tissue of the wearer. Further discussion of the use of such an electrode and counterelectrode configuration may be found in U.S. Patent Application No. 2010/0286590 (entitled: “Iontophoretic Device with Improved Counterelectrode”), which is hereby incorporated by reference herein in its entirety.

In some implementations, when it is desired to further minimize the ability of drug ions to passively transport across barrier layer 211, its polarity may be reversed when the drug is not being delivered. That is, the control circuit may be configured to operate in a “forced inactive” mode and reverse the counterelectrode polarity so that it has the same charge as the drug ions, thus repelling the drug ions away from the patient's skin tissue. Similarly, the polarity of driving electrode 203 may also be reversed by the control circuit in the forced inactive mode, thus enhancing the repulsive effect of barrier layer 211 by attracting the drug molecules towards driving electrode 203 (and hence away from the wearer's skin tissue) by virtue of having the opposite charge as the drug ions. This may be done at a very low power to preserve battery life. This mode of operation may be referred to as forced inactive mode, and the control circuit is configured to be switched to this forced inactive mode to apply these potentials. The term “forced inactive” is used to denote this mode because patch 100 is inactive for delivering the drug, but electrical force is being used to enhance the drug delivery prevention.

In some embodiments, the reversed potentials in this forced inactive mode may be applied to the driving electrode and counterelectrode at predetermined intervals, such as in pulses in accordance with a predetermined duty cycle or a continuous interval. This is done to minimize the energy drawn in the inactive mode. Advantageously, the two electrodes when charged will drive the molecules towards driving electrode 203 and away from counterelectrode 211 and the wearer's skin tissue. Because passive migration back towards the skin will happen rather slowly, the reversed potentials can be pulsed or intermittently applied to offset that passive migration. Thus, a continuous current draw may not be necessary in the inactive mode. In some embodiments, counterelectrode 211 may be used irrespective of whether it functions as a barrier layer. That is, counterelectrode 211 located opposite driving electrode 203 with drug reservoir 205 therebetween may be used to minimize or eliminate the flow of current into the user's skin. In such an embodiment, counterelectrode 211 need not cover the entire bottom surface of the drug reservoir. For example, counterelectrode 211 may have an annular configuration. Other construction or configurations may be used.

In some implementations control portion 103 may include a spacer layer 223 located generally around the perimeter of the bottom surface of base layer 201 in control portion 103. As spacer layer 223 is located around the perimeter of base layer 201 on control portion 103, spacer layer defines an opening 225, wherein battery 217, microprocessor 219, and portions of circuitry 221 are located or housed. In some implementations, spacer layer 223 may be constructed of a foam material so as to provide cushioning and protection of the electrical components housed therein. In some implementations, the bottom surface of spacer layer 223 may be covered by a protective layer 227, which may cover opening 225 so as to further protect the electrical components housed therein. In some implementations, opening 225 or other portion of control portion 103 may house an indicator light 229, which may provide an indicator of whether battery 217 has enough charge to operate patch 100 or to indicate other information. In some implementations, a hole or window may be provided in protective layer 227 so as to enable indicator light 229 to be seen.

FIG. 2B illustrates a portion of patch 100, including a bottom portion of base layer 201, driving electrode 203, battery 217, microprocessor 219, circuitry 221, and other elements. In some implementations, patch 100 may include a connector 233 that is connected to the control circuit by circuitry 221 but electrically isolated from driving electrode 203. Connector 233 serves to electrically connect counterelectrode 211 with the control circuit when the various layers of patch 100 are assembled.

In some implementations, patch 100 may be activated through two modes: 1) an automatic activation and 2) a manual activation. FIGS. 2B and 2C illustrate sensor electrodes 235 that enable automatic activation by detecting whether or not patch 100 is placed on a person's skin (i.e., via a connection detected between sensor electrodes 235 and barrier layer 211). If a connection is detected, patch 100 is switched into active mode and delivery of drug is commenced. If the patch is removed, sensor electrodes 235 detect this removal and stop delivery of the drug. For additional details on a contact sensor, reference may be made to U.S. Patent Publication No. 2011/0092881, the entirety of which is incorporated herein by reference. Manual activation may include a switch (not illustrated) located on control portion 103, that may be manipulated to turn patch 100 on and off.

The foregoing illustrated embodiments have been provided solely for illustrating the structural and functional principles of the present invention, and should not be regarded as limiting. To the contrary, the present invention is intended to encompass all modifications, substitutions, and alterations within the spirit and scope of the following appended claims.

What is claimed:

1. An iontophoretic drug delivery device for delivering a drug into the tissue of a wearer, the device comprising:
a flexible base layer having an upper surface and a lower surface, the base having at least two portions, including a delivery portion and a control portion, wherein the delivery portion includes:
a driving electrode positioned on the lower surface of the base layer,
a drug reservoir containing a supply of charged drug ions, the drug reservoir located beneath the driving electrode, and
a counterelectrode positioned below the drug reservoir, such that the drug reservoir is positioned between the driving electrode and the counterelectrode,
wherein the a control portion includes a control circuit, the control circuit located on the lower surface of the base layer, the control circuit including at least a power source, wherein the control circuit is operatively connected to the driving electrode and the counterelectrode such that when a potential is applied to the driving electrode of the same polarity as the charge of the charged drug ions and a potential of opposite polarity is applied to the counterelectrode, the charged drug ions are motivated towards the tissue of the wearer when the delivery portion of the base layer is placed with the lower surface facing the tissue of the wearer.

2. The device of claim 1, wherein the delivery portion is configured to be placed on the brow of a wearer.

3. The device of claim 1, wherein the delivery portion and the control portion are separated by a bridge portion that has a width narrower than both the delivery portion and the control portion.

4. The device of claim 1, further comprising an adhesive layer located on a bottom surface of the delivery portion of the base only.

5. The device of claim 1, wherein the power source is a battery.

6. The device of claim 1, wherein the control circuit further comprises a controller that controls the application of electrical potential from the power source to the driving electrode and the counterelectrode.

7. The device of claim 6, wherein the controller includes a microprocessor.

8. The device of claim 6, wherein the controller includes at least one switch.

9. The device of claim 1, wherein the drug reservoir includes a hydrogel.

10. The device of claim 1, wherein said control circuit is switchable to a forced inactive mode wherein the control circuit applies a potential to the counterelectrode of the same polarity as the charge of the charged drug ions and a potential of opposite polarity to the driving electrode, thus transporting the drug ions away from the tissue of the wearer.
